(19) United States
(12) Patent Application Publication
(10) Pub. No.: US 2024/0298966 A1
(43) Pub. Date: Sep. 12, 2024

(54) METHOD AND SYSTEM FOR DETECTING FOOD INTAKE EVENTS FROM WEARABLE DEVICES AND NON-TRANSITORY COMPUTER-READABLE STORAGE MEDIUM

(71) Applicant: SAMSUNG ELETRÔNICA DA AMAZÔNIA LTDA., Campinas (BR)

(72) Inventors: WILLIAM C. ARIZA-ZAMBRANO, Campinas (BR); CARLOS A. CAETANO, Campinas (BR); VINICIUS H. CENE, Campinas (BR); PEDRO GARCIA FREITAS, Campinas (BR); LUIS G. L. DECKER, Campinas (BR); ISMAEL SEIDEL, Campinas (BR); JESIMON BARRETO SANTOS, Campinas (BR); OTÁVIO PENATTI, Campinas (BR); JOANA PASQUALI, Campinas (BR); LUCAS PORTO MAZIERO, Campinas (BR)

(73) Assignee: SAMSUNG ELETRÔNICA DA AMAZÔNIA LTDA., CAMPINAS (BR)

(21) Appl. No.: 18/203,738

(22) Filed: May 31, 2023

(30) Foreign Application Priority Data
Mar. 10, 2023 (BR) 10 2023 004544 8

(51) Int. Cl. A61B 5/00; A61B 5/0205; G16H 20/60; G16H 50/70

(52) U.S. Cl. CPC A61B 5/4866; A61B 5/0205; G16H 20/60; G16H 50/70; A61B 5/1123; A61B 5/681; A61B 2562/0219

(57) ABSTRACT

A system and method of detecting food intake events from wearable devices. The system comprises a signal bank to store physiological digital data signals collected via a wearable device comprising at least one sensor to sense at least one physiological parameter of a user. The system comprises a preprocessor module to process the physiological digital data signals stored on the data signal bank and to create a descriptive representation of the sensed at least one physiological parameter. The system also comprises a feature extractor module, with two parallel function modes, comprising features that are automatically learned while others are analytically derived from the descriptive representation. Thus, the feature extractor is configured to select at least one of the modes. The system also comprises a probability estimator module to determine whether a food intake event of the user occurs based on the extracted features.

### CROSS-REFERENCE TO RELATED APPLICATION(S)

This application is based on and claims priority under 35 U.S.C. § 119 to Brazilian Patent Application No. BR 10 2023 004544 8, filed on Mar. 10, 2023, in the Brazilian Intellectual Property Office, the disclosure of which is incorporated by reference herein in its entirety.

### FIELD OF THE DISCLOSURE

The present invention is related to the machine learning field and data-driven modeling applied to health field applications and is related to a method for classifying food intake events, i.e., a method for recognition of meal sessions detection. More specifically, it describes a method and system to actively perform food intake detection by using some steps that can be grouped into three main stages. Considering the customization of these stages, i.e., activating or deactivating some of their components, the proposed method can be configured to be deployed on various types of devices. It can also be configured to use different types of input signals in order to perform food intake classification tasks. Hence, this invention employs various embodiments and can improve a diversity of health and fitness-related applications such as eating behavior monitoring, session regularity and consistency, food type classification, volume and/or weight estimation, etc.

### DESCRIPTION OF RELATED ART

Food intake is an essential activity for human survival. Moreover, the quality of food intake and eating habits strongly correlate with various health conditions such as diabetes, obesity, and cardiovascular diseases. Eating-related diseases affect not only physical health but also impacts significantly in the economy and in the community. According to the paper “Societal and personal costs of obesity” (by Jacob Jaap Seidell, Experimental and Clinical Endocrinology & Diabetes, 1998), the economic costs to the community, including a diversity of resources destined for the diagnosis and treatment of diseases directly related to obesity, as well as the treatment of obesity itself, also has a societal impact on loss of productivity, disability pensions, etc. More objectively, the cost of lost productivity due to obesity is $12,988.51 annually, as reported in “Wearable food intake monitoring technologies: A comprehensive review” (Tri Vu et al., Computers, 2017).

Eating habits are frequently associated with diverse health conditions. Although there are apps designed to monitor eating habits, manually marking calories and meal-intake events is a tedious and time-consuming task that usually causes a loss of user engagement, precluding long-term monitoring. Although some scientific papers and related efforts exist in autonomous meal detection, those proposed solutions are usually particular and rely on vast computational resources. Thus, a pervasive application can help unfold several dietary guidance solutions that have a long-term impact on the user's health.

In this context, approaches for monitoring and inspecting dietary behavior have been investigated. The first approach included manual reports of dietary intake, generally taken in a diary by the subjects to be analyzed by nutritionists and health experts. Recently, with the pervasive presence of smartphones in human life, innovative note-based dietary applications have been developed, as described in the paper “A review of nutritional tracking mobile applications for diabetes patient use” by (Alaina Darby et al., Diabetes technology & therapeutics, 2016). However, although these smartphone-based annotated approaches offer some advantages over handwritten approaches, it is not practical in many cases since it depends on the user's discipline, and they can be inconvenient in many contexts.

To overcome this problem, automatic food intake monitoring must be more effective. By performing this task automatically, users can be free to conduct their activities naturally, especially food-related activities.

Existing solutions rely on naive adaptations of established activity detection technologies or proposals of neural networks for detecting of meal sessions, that is, whether a subject is eating or not, generally focused on the constraints of the end application, such as sensor modality, sensor position, etc.

Most existing solutions use application-constrained approaches to the development of a meal-intake detection system. By incorporating these constraints, these methods are rarely portable to other scenarios. To the best of our knowledge, there is no general method or system that enables meal-intake detection tasks and can be customizable to be deployed in different scenarios.

Moreover, the advantages of automatic food intake monitoring are reflected in a better user experience, which can improve the quality of diagnostics, easy monitoring by the subjects themselves, and also be analyzed by nutritionists and health experts. During the past decade, many studies have been published proposing diverse approaches to performing automatic food intake monitoring. These approaches employ a variety of devices, comprising acoustic, visual, electroglottography (EGG), electromyography (EMG), inertial (i.e., accelerometer and gyroscope), electrical proximity, plethysmograph (PPG), and piezoelectric sensors. Moreover, with the increased availability of wearable technologies, especially smartwatches, access to these sensors has gotten cheaper every year. Since each of these sensing technologies has its pros and cons, diverse methods have been proposed in the literature to monitor food ingestion. These diverse methods were proposed to specific technologies and sensors, and prior art did not include a general framework for designing food intake detection tasks considering these various sensors and target devices.

Patent document U.S. Pat. No. 5,398,688 (Method, system and instrument for monitoring food intake) describes a method for monitoring food intake for an individual and providing an indication when the food intake exceeds a predetermined allowable amount. While document U.S. Pat. No. 5,398,688 is based on the calculation of a maximum eating time determined empirically, the present invention is based on signal processing and machine learning techniques to determine whether a given subject is eating or not. Moreover, document U.S. Pat. No. 5,398,688 is based on a specific hardware, i.e., a wristwatch having a Doppler ultrasound transducer for placing over the user's radial artery and producing an audible alarm when the individual has been eating for sufficient time. Our invention, on the contrary, describes an information system that can be used as the building block to the tracking and detection of food intake sessions.

Patent document U.S. Pat. No. 6,508,762B2 (Method for monitoring food intake) introduces a system that combines photographic video inputs and a computer to monitor a person's food intake under dietary limitations. In other words, the document U.S. Pat. No. 6,508,762B2 is a computer vision application that takes pictures of food items and correlates these taken pictures with other electronically-stored food pictures to identify the food and, as a consequence, carbohydrates, protein, fat, amino acids, and other constituents of the food. Our present invention, on the contrary, describes a software system that assumes that the input data comes from the subject and not from the food. The present invention is proposed to work with various wearable devices that acquire data from several sources, including acoustic, electroglottography, electromyography, accelerometer, gyroscope, plethysmography, or piezoelectric sensors. Patent document U.S. Pat. No. 6,508,762B2 and this present invention have different approaches and different aims.

Patent document WO2001052718A2 (Diet and activity monitoring device) provides an activity-monitoring device for diet monitoring. The device comprises a timer, a body activity monitor, a consumption notation control to indicate when the subject consumes food, an activity calculator to receive the body level, and a consumption calculator. According to claims of WO2001052718A2, the body activity monitor comprises a heart rate sensor, a motion sensor, a respirator sensor, and/or a GPS device. The monitoring device comprises an audio recorder or a digital camera. From these embodiments, it is noticeable that the patent WO2001052718A2 describes a hardware apparatus while this present invention describes an information system and method to design predictive models for food intake detection using machine learning and signal processing approaches. It is worth emphasizing that this present invention and WO2001052718A2 are not exclusive but complementary, with the proposed invention being able to be used as part of the operating system for an embodiment of WO2001052718A2.

Patent document U.S. Pat. No. 5,233,520A (Method and system for measurement of the intake of foods, nutrients, and other food components in the diet) proposes a computer device integrated with an electronic balance, output display devices, user input elements, a food codes database, and a storage element. The document U.S. Pat. No. 5,233,520A discloses a system and process for determining which food, as well as its nutrients and components. In other words, the document U.S. Pat. No. 5,233,520A describes a computational device for detecting food properties, while this present invention describes a method and system for detecting when a human subject is eating. Therefore, U.S. Pat. No. 5,233,520A and the proposed invention have different aims.

Patent document US20210350920 (Method and apparatus for tracking food intake and other behaviors for providing relevant information feedback) relates to electronic devices that relate to apparatus for using sensors for tracking food intake. The document US20210350920 discloses a processor for analyzing a food intake process and electronic circuits for providing information feedback to the person. Despite this invention and the document US20210350920 presenting similar aims, these documents present dissimilar methods for achieving them. For instance, based on the first to the fourth claim of document US20210350920, the method described in US20210350920 assumes at least one wearable device comprising a camera. The food intake event detection is performed using a bite count, a sip count, an eating pace, or a drinking pace.

On the other hand, the present invention does not assume a camera, but inertial sensors, temperature sensors, bioelectrical impedance sensors, or photoplethysmography sensors. Moreover, based on the fifth to eighth claims of US20210350920, it is realized that the method of US20210350920 requires a projecting light source of at least one wearable electronic device in response to determining the start of the food intake event. Differently, this present invention proposes a method that does not requires light sources to detect the start and end of a food intake session and can be used in low or no-light environments. Furthermore, the document US20210350920 presents a general description of a system that might include detecting, identifying, analyzing, quantifying, tracking, processing, and/or influencing the related intake of food, eating habits, eating patterns, and/or triggers for food intake events, eating habits, or eating patterns. This present invention is focused on a system and method for detecting only food intake events. Therefore, in brief, the present invention and US20210350920 are not exclusive but complementary, with the proposed invention being able to be used as the food intake event subsystem of US20210350920.

Patent document JP2019185405A (Meal detection program, meal detection method, and meal detection device) describes a method and device for detecting meal session times by calculating the difference between the meal start time and end. The document JP2019185405A is based on the effect of meal size on the cardiovascular response to food ingestion and only detects the characteristics of the heartbeat during a meal and a certain size of the meal is ingested. For that reason, the technique described in JP2019185405A is restricted to the acquired heartbeat signal data, and the method is based on the detection of peaks in this signal. On the other hand, the present invention is not restricted to heartbeat data but is general enough to use data collected from inertial, temperature, bioelectrical impedance, or photoplethysmography sensors. Since the proposed invention is based on automatic probability estimation, it does not depend on specific signal waveform characteristics, such as peaks and valleys. Moreover, the present invention includes pre and post-processing steps, which makes the present invention substantially different from document JP2019185405A.

Patent document U.S. Pat. No. 10,702,210B2 (Meal intake detection device, meal intake detection method, and computer-readable non-transitory medium) discloses a computational device composed of a central processing unit, a random access memory, a display device, an acceleration sensor, a storage device, and a heartbeat measuring sensor. The device described in U.S. Pat. No. 10,702,210B2 is configured to detect a rise of heart rate in order to extract possible food intake session from the instant in time when a rise of the heart rate is detected, to the time when the heart rate decreases to a predetermined value. On the other hand, the present invention is not restricted to heartbeat data being general enough to use data collected from inertial, temperature, bioelectrical impedance, or photoplethysmography sensors. Thus, our invention is agnostic to specific waveform features and can autonomously estimate the probability that the user is in a meal intake session. Moreover, our invention also encapsulates pre and post-processing steps, highlighting the different approach compared to U.S. Ser. No. 10/702,210B2.

Patent document EP3231362A1 (Meal intake estimation program, meal intake estimation method, and meal intake estimation device) describes a method and device that causes a computer to execute a process comprising of heart rate time-series acquisition, peak-based feature quantifying, meal session identification, and meal time estimation. Therefore, the document EP3231362A1 presents a method that identifies the presence of a meal based on the peaks of the heart rate signal. On the other hand, the present invention is not restricted to heartbeat data being general enough to use data collected from inertial, temperature, bioelectrical impedance, or photoplethysmography sensors. Our invention relies on automatic probability estimation that the user is in a food intake session or not, from a data-driven approach. Moreover, the present invention comprises the pre and post-processing steps, which makes the present invention substantially different from EP3231362A1.

Patent document U.S. Pat. No. 9,536,449B2 (Smartwatch and food utensil for monitoring food consumption) relates to a device and system for monitoring a person's food consumption comprising a wearable sensor that automatically collects data to detect eating events a Smart food utensil, probe, or dish that collects data concerning the chemical composition of food which the person is prompted to use when an eating event is detected, and a data analysis component that analyzes chemical composition data to estimate the amounts and types of food, ingredients, calories, etc. In document U.S. Pat. No. 9,536,449B2, a smartwatch can be used as the wearable sensor, but other devices can be used in different embodiments, such as a smart bracelet. Our present invention is different from document U.S. Pat. No. 9,536,449B2 because the present invention does not collect data from the dish or chemical composition data nor requires smart food utensils. The present invention describes a method and information system for automatically detecting when a person is eating or not. The present invention only requires a wearable device, such as a smartwatch, without additional devices.

Moreover, since document U.S. Pat. No. 9,536,449B2 does not detail how the wearable sensor performs the automatic detection, the present invention can be used as part of the operating system running in the wearable device mentioned in U.S. Pat. No. 9,536,449B2. Therefore, in short, the present invention and U.S. Pat. No. 9,536,449B2 are not exclusive but complementary, with the proposed invention being able to be used as the eating event detecting subsystem of U.S. Pat. No. 9,536,449B2. Our invention can be adapted to be used in a smart utensil where temperature and/or inertial sensors (among others) can be used to autonomously estimate the probability that the user is in a meal intake session.

Patent document U.S. Pat. No. 10,980,477B2 (Electronic device and method for providing digestibility on eaten food) outlines a general device and its various embodiments, including a biometric sensor, an output device, and a processor operatively coupled with both devices. The main idea is to obtain meal intake information while using the biometric sensor to estimate the digestibility of the ingested food. Therefore, in summary, the document U.S. Pat. No. 10,980,477B2 presents a device and method for determining the digestibility of food by the user, while the present invention proposes a method and system to detect when the user is ingesting food. For that reason, document U.S. Pat. No. 10,980,477B2 and the proposed invention have different aims. The U.S. Pat. No. 10,980,477B2 relies on a biometric sensor waveform and pre-specified non-biometric information via the communication circuitry to estimate meal consumption. Contrastively, this invention is based on autonomous probability estimation. It does not depend on specific signal waveform characteristics, such as peaks and valleys, using solely data-driven features, which, along with the pre and post-processing stages, makes the present invention substantially different from U.S. Pat. No. 10,980,477B2. It is worth mentioning that the present invention and U.S. Pat. No. 10,980,477B2 are not exclusive but complementary, with this invention being able to be used as the food intake event subsystem of U.S. Pat. No. 10,980,477B2.

The paper “Deep Learning for Intake Gesture Detection From Wrist-Worn Inertial Sensors: The Effects of Data Preprocessing, Sensor Modalities, and Sensor Positions” (by Heydarian, H., Rouast, P. V., Adam, M. T., Burrows, T., Collins, C. E., & Rollo, M. E, IEEE Access, 2020) proposes the use of a deep neural network to process collected inertial data and detect characteristic hand movements associated with intake gestures. This paper, although having the same aim of providing food intake detection, is different from the present invention. This invention does not depend on a neural network architecture to perform meal intake detections. Although such a method is possible, it is not a requirement for the second stage of this invention.

The paper “Food Intake Actions Detection: An Improved Algorithm Toward Real-Time Analysis” (by Gambi, E., Ricciuti, M., & De Santis, A., Journal of Imaging, 2020) presents an application aiming to monitor the food intake actions of people during a meal using a Kinect device, being a computer vision application. On the other hand, this invention describes a method and system to predict food intake using inputs from wearable sensors. Therefore, although the paper and this invention aim for similar purposes of detecting food intake actions, the approaches, scope, and algorithms in these two documents are quite different.

The paper “Modeling Wrist Micromovements to Measure In-Meal Eating Behavior From Inertial Sensor Data” (by Kyritsis, K., Diou, C., & Delopoulos, A., IEEE journal of biomedical and health informatics, 2019) presents an algorithm for automatically detecting the in-meal food intake cycles using the inertial signals (acceleration and orientation velocity) from an off-the-shelf smartwatch. Despite this paper and the present invention sharing similar aims, the paper proposes an algorithm for measuring the in-meal eating behavior by performing temporal localization of the intake moments (i.e., bites) using inertial signals. In contrast, the present invention describes a general method and system for designing food intake detection tasks considering various sensors from wearable devices. To perform the food intake session detection, the paper uses five specific wrist micromovements to model the series of actions and conduct the detection in two steps. In the first step, the windows of raw sensor streams are processed, and the micromovement probability distributions are estimated using a convolutional neural network. In the second step, a long short-term memory network is used to capture the temporal evolution and classify sequences of windows as food intake cycles. On the other hand, the proposed invention comprises the pre and post-processing steps further to the autonomous probability estimation. Therefore, the present invention and the referred paper are not exclusive but complementary, with the method described in this paper being able to be used as a neural network subsystem or an auxiliary component of the proposed invention.

The paper “Detecting Meals In the Wild Using the Inertial Data of a Typical Smartwatch” (by Kyritsis, K., Diou, C., & Delopoulos, A., 41st Annual International Conference of the IEEE Engineering in Medicine and Biology Society (EMBC), 2019) proposes a two-step method to detect meals in environments without control (i.e., without a protocol to be followed during meals), using the inertial data (acceleration and orientation velocity) from commercially available smartwatches. In the first step, the raw inertial signals are processed using an End-to-End Neural Network to detect the bite events throughout the meal. In the second step, the resulting bite detections are processed using signal processing algorithms to obtain the final meal start and end timestamp estimates. On the other hand, the proposed invention is composed of the pre and post-processing steps in addition to the autonomous probability estimation stage and can detect bite-agnostic meal intakes composed of liquids. Therefore, the present invention and the referred paper are not exclusive but complementary, with the method described in this paper being able to be used as a neural network subsystem or an auxiliary component of the proposed invention.

The paper “An Intelligent Food-Intake Monitoring System Using Wearable Sensors” (by Liu, J., Johns, E., Atallah, L., Pettitt, C., Lo, B., Frost, G., & Yang, G. Z, 9th international conference on wearable and implantable body sensor networks, 2012) present a wearable sensor platform that autonomously provides detailed information regarding a subject's dietary habits using a microphone and a camera and is worn discretely on the ear. On the other hand, the present invention does not target visual (camera) or sound (microphone) data but is general enough to use data collected from inertial, temperature, bioelectrical impedance sensors, or photoplethysmography sensors. Moreover, the proposed invention describes a general method and system for designing food intake detection tasks considering various sensors from wearable devices. Moreover, the present invention comprises the pre and post-processing steps and an autonomous probability estimation detector, which makes the present invention substantially different from the paper.

The paper “AutoDietary: A Wearable Acoustic Sensor System for Food Intake Recognition in Daily Life” (by Bi, Y., Lv, M., Song, C., Xu, W., Guan, N., & Yi, W., IEEE Sensors Journal, 2015) introduces a wearable system to monitor and recognize food intakes in daily life by proposing an embedded hardware prototype to collect food intake sensor data, which is highlighted by a high-fidelity microphone worn on the subject's neck to precisely record acoustic signals during eating in a non-invasive manner. The acoustic data are pre-processed and then sent to a smartphone via Bluetooth, where food types are recognized. This paper is different from the proposed invention in many folds. The paper and the present invention have different aims, i.e., the paper address the problem of recognizing food type and classifying the type intake (i.e., liquid, solid food, etc.). On the other hand, the proposed invention aims to recognize whether a human subject is eating for a period of time. Moreover, the techniques employed to achieve their corresponding aims are pretty distinctive. In particular, the paper uses hidden Markov models to identify chewing or swallowing events, which are then processed to extract their time/frequency-domain and nonlinear features, and a lightweight decision-tree-based algorithm is adopted to recognize the type of food. The present invention, as stated before, does not define explicit feature models but employs an autonomous probability estimation.

The paper “Implementing Real-Time Food Intake Detection in a Wearable System Using Accelerometer” (by Ghosh, T., Hossain, D., Imtiaz, M., McCrory, M. A., & Sazonov, E., IEEE-EMBS Conference on Biomedical Engineering and Sciences (IECBES), 2021) describes a real-time food intake detection method for wearable sensor systems using accelerometer sensors. Despite this paper and the present invention having the same aim, their algorithms and manner of achieving their objectives are noticeably different. The paper employs a wearable sensor system to monitor food intake through non-invasive jaw movement. Masticatory muscle activation, such as temporal muscle movement, is captured using a sensor system. The paper proposes an “Automatic Ingestion Monitor” (AIM) attached to the temple of eyeglasses to detect temporalis muscle activation during food crushing and to suck off liquids.

The accelerometer sensor is assembled in AIM and is used to detect chewing. So, in summary, the wearable device proposed in the paper is an eyeglass with an accelerometer that collects data from facial muscles. These accelerometer signal data are then processed to extract six features: sum, the sum of mean absolute difference, range, number of mean-crossing, the number of slope sign changes, and wavelength. These features are used as input in a decision tree algorithm that classifies whether the user is consuming food. On the other hand, the present invention does not describe a specific device but proposes a method and system for designing food intake detection tasks considering various sensors and deployable devices. For instance, the invention could be combined with the method proposed in the paper in place of the feature extractor and decision tree algorithm. In the case of this combination, the device described in the paper could be used to collect the signal data, and the method proposed in this invention could be used to process this data to determine food intake sessions. Therefore, it is worth mentioning that the present invention and the paper are not exclusive but can be used complementary, with the proposed invention being able to be used as the food intake event subsystem of the device specified in the paper.

Therefore, the prior art lacks a solution that can detect food intake events with autonomous probability estimation and that can be applied with different types of sensors.

### SUMMARY OF THE INVENTION

The proposed invention aims to introduce a novel system and method for food intake session detection. The system is general enough to be deployed in different health and fitness applications and customized according to the available sensors or even using sensor fusion techniques. This customization is possible thanks to the modularized nature of the proposed method, composed of three main stages with specific and flexible modules that can be configured to accommodate possible trade-offs related to sensors and power restrictions considering wearable devices.

The proposed invention aims to present a general and flexible model to produce customizable embodiments that are not restricted to a specific application and can be customizable to achieve the target application.

The proposed invention aims to detect meal intake events using different pre-processing, modeling, and post-processing configurations. These configurations can be adapted to use a variety of sensor modalities, sensor positions, and computational resources.

Another objective of the present invention is to bring a novel competitive way of detecting food intake. The system proposed in this invention solves the problem of designing predictive pipelines for food intake and associated applications, such as autonomous dietary guidance, which remain an unsolved problem in the literature. It relies on three main steps that can be used as the building block to track eating-related phenomena and are flexible to produce different predictive models, depending on the aimed requirements and hardware constraints.

Aiming at achieving the objectives above, the present invention refers to a system for detecting food intake events from wearable devices, the system comprising: a signal data bank for storing physiological digital data signals collected via a wearable device comprising at least one sensor for sensing at least one physiological parameter of a user. The system comprises a preprocessor module for processing the physiological digital signals stored on the signal data bank to create a descriptive representation of the sensed at least one physiological parameter. Moreover, the system comprises a feature extractor module comprising an automatically learned feature extractor and an analytical feature extractor for extracting features from the descriptive representation, wherein the feature extractor module is configured to select at least one of the automatically learned feature extractor or the analytical feature extractor. In addition, the system comprises a probability estimator module for determining whether the user is in a food intake event based on the extracted features.

Moreover, the present invention refers to a method for detecting food intake events from wearable devices, the method comprising the steps of:
storing physiological digital data signals in a signal data bank, wherein the physiological digital data signals are collected via a wearable device comprising at least one sensor for sensing at least one physiological parameter of a user;
processing, with a preprocessor module, the physiological digital signals stored on the signal data bank to create a descriptive representation of the sensed at least one physiological parameter;
extracting, with a feature extractor module, features from the descriptive representation, the feature extractor module comprises an automatically learned feature extractor and an analytical feature extractor and is configured to automatically select at least one of the automatically learned feature extractor or the analytical feature extractor; and
generating, with a probability estimator module, an estimated probability to determine whether the user is in a food intake event based on the extracted features.

The present invention is also related to a non-transitory computer-readable storage medium adapted for performing the method for detecting food intake events from wearable devices.

### BRIEF DESCRIPTION OF THE DRAWINGS

The invention is explained in greater detail below and references the drawings and figures attached herewith, when necessary. Attached herewith are the following:

FIG. 1 shows a components overview of the system according to an embodiment of the present invention.

FIG. 2 shows elements of the preprocessor according to an embodiment of the present invention.

FIG. 3 shows a feature extractor in detail according to an embodiment of the present invention.

FIG. 4 shows a probability estimator according to an embodiment of the present invention.

FIG. 5 shows a postprocessor according to an embodiment of the present invention.

FIG. 6 shows a preferred embodiment of the present invention.

FIG. 7 shows an alternative embodiment of the present invention.

FIG. 8 shows the qualitative results of the proposed invention on the SRR dataset an embodiment of the present invention.

FIG. 9 shows prediction errors associated with transition states an embodiment of the present invention.

FIG. 10 shows the relation between errors achieved with and without post-processing an embodiment of the present invention.

FIG. 11 shows corrections of erroneous prediction when using the post-processing component an embodiment of the present invention.

### DETAILED DESCRIPTION OF THE INVENTION

The present invention relates to a method for classifying and recognizing food intake sessions using machine-learning techniques. This invention actively extracts behavioral features through various processing steps, including a wearable device assembled with a set of sensors that collect a bank of signals. Signals in the bank of signals are processed in a pre-processing component to be used in a feature extraction component. This pre-processing component is composed of several parts that transform the raw sampled signals into a descriptive representation, in a manner that the feature extractor can extract meaningful information. The feature extractor initiates from an original set of pre-processed data and builds features designed to be informational and non-redundant, simplifying the subsequent learning and generalization steps in the estimation component. The estimation component, in turn, transforms the features into a probability estimation. This estimation refers to the probability that a given subject is eating or not. Moreover, this probability estimation can be fine-tuned by a post-processing component that implements techniques to improve the final eating/not-eating prediction.

In this document, for easier understanding, we detail all the minor steps and components of the proposed invention. Furthermore, for the sake of simplicity, we provide some scenarios where the invention can be applied. Such scenarios, present as embodiments of this document, do not restrict the scope and applications of the proposed application. On the contrary, the proposed method by the present invention can be applied to various electronic communication devices, such as smartphones, smartwatches, smart jewelry, fitness trackers, smart clothing, and implantable chips, among others. Our invention is flexible enough to be configured to operate with meager resources and enable a lightweight solution, able to be embedded in resource-constrained devices with minimal energy consumption impact. Moreover, another substantial advantage of the invention is that no hardware changes are required to implement the proposed method in existing devices.

To achieve this, the present invention proposes a system for detecting food intake events from wearable devices; the system comprises a signal data bank for storing physiological digital data signals collected via a wearable device comprising at least one sensor for sensing at least one physiological parameter of a user. The system comprises a preprocessor module for processing the physiological digital signals stored on the data signal bank to create a descriptive representation of the sensed at least one physiological parameter. Moreover, the system comprises a feature extractor module comprising an automatically learned feature extractor and an analytical feature extractor for extracting features from the descriptive representation, wherein the feature extractor module is configured to select at least one of the automatically learned feature extractor or the analytical feature extractor. In addition, the system comprises a probability estimator module for determining whether the user is in a food intake event based on the extracted features.

Considering the human behavior related to eating habits (i.e., people produce similar movements during food intake activities), the present invention proposes a method to extract, process, and classify patterns in this behavior to determine whether a person is in an eating action at a specific time, as illustrated in FIG. 1. During an eating session 101, a person tends to perform particular movement patterns with the hand to manipulate cutlery and bring food from the dish on the table to the mouth, among others. These movement patterns can be characterized when an appropriate device, such as a wearable device 102, collects convenient data describing such patterns. The convenient data for this application are called signals, functions that convey information about a phenomenon. These signals are collected using appropriate sensors assembled in the wearable device 102 for sensing at least one physiological parameter of a user. In the illustrative embodiments, the wearable device 102 is shown as a smartwatch that is attached to the user's wrist and is capable of collecting physiological data. However, in different embodiments, the device 102 could be one or many devices, such as smartphones, smartwatches, smart jewelry, fitness trackers, smart clothing, and implantable chips. Still, the user's wearable device can be a wearable sensor or other wearable devices such as earphones, or smartphones in contact with the user's body.

To coordinate and allow the interaction and communication between the several blocks or components proposed in this invention, some mechanisms are likewise required to control the desired workflow and/or data flow. We can understand workflow control as the mechanism used to orchestrate the processes or components activated or used in a specific framework configuration. Similarly, we can understand the data flow as how the data is shared through the different components or stages described in this invention. Specifically, this invention uses the Fuser and Selector components as the work and data flow control mechanisms.

The Fuser's responsibility is to aggregate data from multiple sources into a single data stream. This data can be incorporated in multiple ways given the usage context of the Fuser: (i) the multiple inputs can be concatenated together and then transmitted to the output, or (ii) a data aggregation function can be used to merge the multiple input data into a single output, or (iii) the Fuser can select data for one or more sources using a data selection function.

Complementarily, the Selector component can multiplex and/or demultiplexing various components and data paths in our invention. The Selector reads a configuration state and translates it as the activation or deactivation of one or several pipeline components. Once a Selector selects a component, it is connected to the pipeline, and its respective data inputs are used and propagated through the further stages of the pipeline. The inputs and outputs from unchosen components are ignored, and no further processing is performed.

The proposed invention adopts a signal bank 103 to store signals collected via a wearable device 102 for extracting descriptive information about the user wearing the device. A diversity of signals can be stored in the signal bank. Examples of these signals include but are not restricted to pulsatile signals 104, inertial sensor signals 105, skin temperature sensors 106, and much more. The proposed system has a flexible, modular, and configurable nature that enables a general implementation with various wearable devices that acquire signals from acoustic, electroglottography, electromyography, accelerometer, gyroscope, plethysmography, piezoelectric sensors or bioimpedance sensors.

The present invention is not restricted to single or multiple signals. In other words, the proposed method can be used to predict if a user is in an intake food session by using a single signal or multiple signals according to convenience. Pertinent information can be extracted if a Preprocessor 107 is appropriately configured. For instance, if a single signal is available, the preprocessor 107 must be configured to extract the information as best as possible. On the other hand, if multiple signals are available, the preprocessor 107 must be configured to fuse the signals data to produce a convenient descriptive representation for the Feature Extractor 108. In exemplary embodiments, the preprocessor 107 may perform some tasks such as: (i) resampling of the input signal, given that the input signal is at 100 Hz, the preprocessor 107 can be configured to resample it to 200 Hz; (ii) apply normalization functions to the input signal; (iii) apply signal filtering functions. Thus, the output of the preprocessor 107 consists of the signal processed by these functions configured according to each application. The number of input signals will only affect the final size of the representation. If there are more input signals, there will be a larger output. However, the pre-processing functions can be applied regardless of the input size, that is, the number of signals.

The Feature Extractor 108 is the component responsible for preparing input features for the Probability Estimator 109, which is responsible for estimating the probability of the person using the device being in an eating session. The binary prediction (i.e., whether the person is eating or not) is based on this probability. However, before the binary prediction, the probability estimation can be further improved by the Postprocessor component 110. The Postprocessor component 110 adopts a set of techniques that take the probabilities curve (i.e., the probabilities estimated by the Probability Estimator 109 during a period, forming a time series) as input and outputs another more adequate probability curve. Finally, after computing the best probability estimation possible, the final prediction is reported to the Logbook component 111. The Logbook Component 111 can be used to record states, meal intake events, or eating conditions applicable to complex applications that operate them.

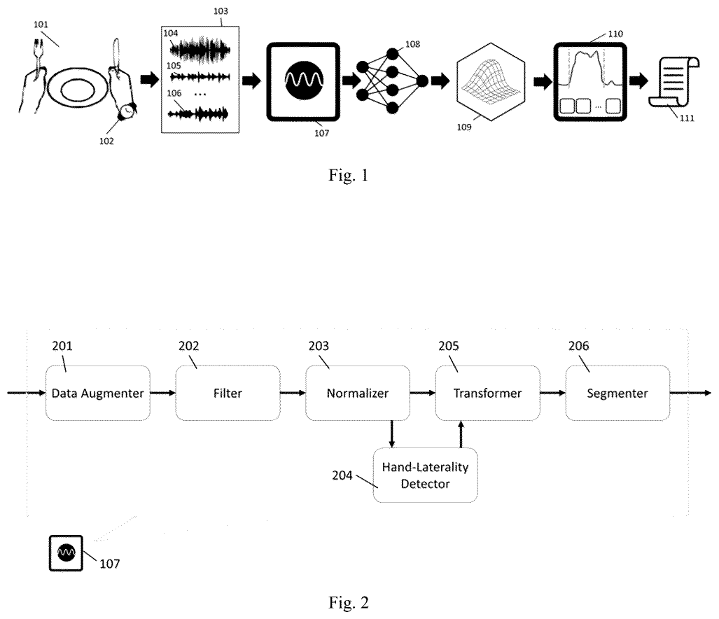

The Preprocessor component 107 is detailed in FIG. 2. It is formed by a pipeline composed of sub-components, including Data Augmenter 201, Filter 202, Normalizer 203, Hand-Laterality Detector 204, Transformer 205, and Segmenter 206. All these components can be used or not, depending on the need. For instance, if a data amount sampled in the signal bank, 103, is sufficiently representative to describe the food intake phenomenon, data augmenter 201 can be deactivated or omitted from an implementation, even if it can boost the robustness of the system. Filter 202 and Normalizer 203 are responsible for adjusting the signal coming from a sensor (or augmented by the data augmenter 201) into a convenient numerical representation that can be used in the following stages of the proposed pipeline. The Filter component 202 is implemented for noise reduction, data smoothing, and to suppress unwanted signal frequencies that do not contribute to the prediction model or impair the performance of the following components. The Normalizer 203 adjusts the signal samples into an appropriate range (e.g., [0, 1], [−1, 1], or [0, 255]) to be processed by the other components without risks of underflow or overflow. Similar to the data augmenter 201, the filter component 202 and normalizer 203 can be deactivated or omitted according to the implementation of this invention. For example, if the sensors already sample the signals with a convenient waveform and use the same range required by the other components, there is no need to perform filtering or normalizing operations.

The Hand-Laterality Detector component 204 is used when the proposed invention is implemented in a hand, wrist, or arm wearable device. In these scenarios, it may be convenient to detect in which hand the device is being used so that the overall model behaves more smoothly according to the hand of the user. Considering an application based on wearable devices with inertial sensors worn on the hand, wrist, or arm. The lateral detection can be performed based on the characteristic profile of upper limb movements. For example, abduction and adduction movements on each side are mirrored. By analyzing the density distribution of the measured signals and comparing it with a known distribution, the laterality of the device can be assessed. The laterality detection can be implemented using other devices or applications, e.g., an application considering video cameras, which can identify the key points of an anthropomorphic body and determine laterality by analyzing the relative positions of identified key points.

One of the examples of the usage of Hand-Laterality Detector 204 is to communicate to the Transformer 205, which will, in turn, transform data that vary according to hand laterality (e.g., inertial data collected by sensors such as accelerometers and gyroscopes) into a convenient representation. Examples of convenient transformations performed by the transformer 205 in combination with the Hand-Laterality Detector 204 include, but are not restricted to, hand mirroring (by converting left-handed data to right-handed data) and hand-stacking (stacking both hands data). Moreover, transformer 205 can also perform additional data transform operations that do not depend on the Hand-Laterality Detector 204, such as signal rectification (i.e., rectifying the input signal by mirroring negative components into positive values). Finally, Segmenter 206 is in charge of subdividing the sampled signal into segments, and these segments are subdivided into smaller parts called windows. These segments are parts of the signal buffered on a memory (e.g., the last two hours of sampled data), while windows are smaller blocks (overlapping chunks) slid over the segment. The windows are used as the inputs of components Feature Extractor 108 and postprocessor component 110.

The Feature Extractor 108 gets the data prepared by Preprocessor 107 as input and transforms it into a suitable representation using machine learning and/or analytical techniques. More specifically, a component called Selector 301 selects if Preprocessor 107 will use the Automatically Learned Feature Extractor 302, the Analytical Feature Extractor 303, or both. Depending on the system configuration, the input data will be transmitted from the Selector 301 to the Automatically Learned Feature Extractor 302, the Analytical Feature Extractor 303, or replicated to both. The Automatically Learned Feature Extractor 302 learns how to extract representative features from data using neural networks. Examples of neural network architectures that can be used in place of the Automatically Learned Feature Extractor 302 include, but are not restricted to, the models described in the papers “DanHAR: Dual Attention Network For Multimodal Human Activity Recognition Using Wearable Sensors” (by Gao, W., Zhang, L., Teng, Q., He, J., & Wu, H., Applied Soft Computing, v. 111, p. 107728, 2021), “Deep Learning for Intake Gesture Detection From Wrist-Worn Inertial Sensors: The Effects of Data Preprocessing, Sensor Modalities, and Sensor Position” (by Heydarian, H., Rouast, P. V., Adam, M. T., Burrows, T., Collins, C. E., & Rollo, M. E., IEEE Access, v. 8, p. 164936-164949, 2020), “Human activity recognition based on smartphone and wearable sensors using multiscale DCNN ensemble” (by Sena, J., Barreto, J., Caetano, C., Cramer, G., & Schwartz, W. R., Neurocomputing, v. 444, p. 226-243, 2021), “Smartwatch-based eating detection: Data selection for machine learning from imbalanced data with imperfect labels” (by Stankoski, S., Jordan, M., Gjoreski, H., & Luštrek, M., Sensors, v. 21, n. 5, p. 1902, 2021), etc.

The Analytical Feature Extractor 303 can extract multiple analytical features. Similar to Selector 301, the Analytical Feature Extractor 303 also has a selector 304 that chooses which techniques will be used. The Selector 304 can select Entropy 305, Statistical 306, or Complexity 307 feature extractor.

The entropy feature extractor 305, for example, may apply entropy methods that are used to capture the changes in the intrinsic structures of the sensor, such as approximate entropy, entropy sample, Fuzzy entropy, permutation entropy, weighted permutation entropy, and incremental entropy, such as seen in reference Ding, Fengqian, and Chao Luo. 2019. “The Entropy-Based Time Domain Feature Extraction for Online Concept Drift Detection” Entropy 21, no. 12: 1187. https://doi.org/10.3390/e21121187.

The statistical feature extractor 306 may extract statistical features such as mean, standard deviation, mean absolute deviation, minimum value, maximum value, the difference between maximum and minimum values, median, median absolute deviation, counting negative values, counting positive values, number values above average, number of peaks, asymmetry, average resulting acceleration, among others.

The complexity feature extractor 307 may measure the interrelations between the different values in a time series (the sensor input data). For example, the following techniques can be cited: Kurtosis, the “tail” of the probability distribution, and Skewness and the asymmetry of the probability distribution.

The feature extractors 305, 306, and 307 can be used solely or combined, depending on how the Selector 304 was configured, and the input data will be transmitted or replicated to the active components. The Fuser component 308 combines the outputs of 305, 306, and 307. It transmits this combination to a second Fuser component, 309, which fuses the features produced by the Automatically Learned Feature Extractor 302 and the Analytical Feature Extractor 303 to generate the final output of feature extractor 108. Selector 301 and Fuser 309 are symmetric, i.e., if Selector 301 does not select a given feature extractor, the Fuser 309 will only retransmit the features to the next component.

The Probability Estimator 109 component has two main components: the Dominant Hand Detector 401 and the Meal Session Probability Calculator 402. The Dominant Hand Detector 401 detects the user's handedness and informs it to the Meal Session Probability Calculator 402. A method for identifying hand dominance using wearable inertial sensors is similar to the lateral detection method. It involves comparing the density distribution of the measured signals with a known distribution. In the case of hand dominance, it is helpful to consider the total energy of the captured signal, as the dominant hand tends to be more active than the other hand. The distribution contrastive process can be implemented using a distance criterion or learning a classifier directly from the data. Depending on the user's dominant hand, the Meal Session Probability Calculator 402 estimates the probability using the most suitable model, i.e., it selects the model for the laterality detected by the Hand-Laterality Detector 204 considering the laterality detected by the Dominant Hand Detector 401.

The Postprocessor component 110 is composed of two main sub-components: Thresholding-Based Postprocessor 501 and Heuristic-Based Postprocessor 502. Thresholding-Based Postprocessor 501 has a threshold algorithm Selector 503 that chooses if the Single-reference thresholding algorithm 504, Hysteresis threshold algorithm 505, or both are used. Depending on what is selected, a Fuser component 506 combines the outputs of 504 and 505 and transmits them to the following pipeline stages. Heuristic-Based Postprocessor 502 also has a Selector component 507 that selects one or more heuristics, including Minimal Meal Time 508, which is the minimum time considered as a meal session, and Onset Time Compensator 509, which aims to compensate for the delay time that takes the estimator to change the system from a non-eating state to an eating state, Offset Time Compensator 510 which also compensates the time of the state changing from eating to non-eating. Session Cuts Removing 511 that reduces the inner probability gaps of a meal session. The Fuser component 512 combines the outputs of the selected heuristics 508, 509, 510, and 511 and transmits the fused output to the following depicted stages of the pipeline.

The Change Point Detector 514 is an additional component that tries to identify times when the probability distribution of an eating event changes. It aims to improve the eating/non-eating classes' transitions, enhancing the predictions to determine more precisely when a meal-intake session started and ended. This method is based on the derivative of the probability curve. A peak in the derivative curve means that a transition state has occurred, a change point. A group of peaks within a certain proximity to each other determines an analysis region. The change point detector method applies a function for every analysis region detected, updating the transition points and improving the event detection accuracy.

Finally, depending on the system configuration defined at the implementation, the selected components of the postprocessor 110 are activated by Selector 513. The Fuser 515 is responsible for fusing all corresponding output predictions into an enhanced probability curve estimation, and this enhanced estimation is transmitted to the logbook component 111, which reports the binary predictions.

### Preferred Embodiment

FIG. 6 discloses an illustrative embodiment of the invention when a given wearable device (e.g., smartwatch) has the computational capacity to run components to perform all steps of the proposed invention. In this embodiment, a person 101 wearing a wearable device 102 needs only the smart device to run the whole proposed method. The wearable device 102 contains all requirements 601 for running the proposed method. Device 102 contains a sensor 602, a processor 603, and a memory 604 with enough computational capacity to run all the steps of the proposed method 605. While the complete pipeline of this invention method is performed, the meal intake detection performed along the time is saved in the Meal Event Buffer 606. This buffer 606 can be consumed by a variety of applications 607 that run inside the device.

In this embodiment, the Preprocessor 107 must be configured to provide all the relevant sensor signals present in the Smart Device 102 to the Feature Extractor 108. Given that in this scenario, the computational and energetic resources are limited, it may be more interesting to configure Selector 301 from the Feature Extractor 108 to route the data only to the Automatic Learned Feature Extractor 302 or to the Analytical Feature Extractor 30), given that the feature extraction by multiple algorithms may be very resource consuming. The Fuser 309 should be configured to replicate the data from the selected feature extractor to its output.

### Alternative Embodiments

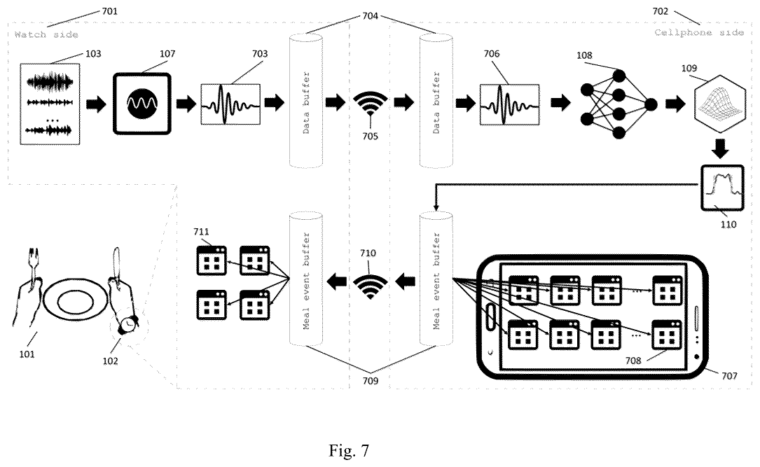

FIG. 7 discloses another exemplary embodiment of the present invention. This embodiment can alleviate the hardware requirements of wearable devices, enabling the method to run on platforms with severe memory, processing, and power restrictions. In this case, the wearable device (e.g., smartwatch) acts as a sensor bank and display. All computationally expensive components and steps are performed in a smartphone device. In this embodiment, a person 101 wearing a smart device also needs a cellphone, both able to communicate with each other. The system is divided into two significant parts in this setup: the Watch side 701 and the Cellphone side 702. The Watch side 701 is responsible for collecting (sensing), preparing (pre-processing) the data, and displaying the final results.

The signal collected by the sensors of the wearable device 102 is stored in the signal bank 103 and processed with the Preprocessor 107 presented in this invention. The result of the preprocessor 107 is a convenient data representation 703 of the information that can be used as input in the Feature Extractor 108. The data 703 is compressed and stored in a Data Buffer 704, a data structure synchronized between the two devices over a network 705. After transmission, the received data 706 is used as input in the Feature Extractor 108, and the subsequent steps described in this invention are performed, i.e., Probability Estimator 109, and Postprocessor 110, among others. After all steps, the prediction results are stored in the Meal Event Buffer 709, another data structure synchronized between the two devices over a network 710. The synchronized buffer 709 enables access to prediction for applications running in the cellphone 708 and applications running in the smart device 711.

Since, in this embodiment, there are fewer computational and energetic restricted on the computationally expensive steps, the Feature Extractor 108 component can be configured in a manner that optimizes the pipeline to a better efficiency: both Selectors 301 and 304 can be configured to propagate the input data to more than one feature extractor. Selector 301 can select both the Automatic Learned Feature Extractor 302 and the Analytical Feature Extractor 303; meanwhile, the Selector 304 can be configured to propagate the input to a combination of the Complexity 307, Statistical 306, and Entropy 305 feature extractors. Consequently, the Fusers 308 and 309 must be configured to take all the features extracted from the previous components and concatenate them in the output.

We evaluate the effectiveness of the present invention on the in-the-wild meal intake detection task, which deals with measuring the performance of the meal detection method using real-world recordings (i.e., no controlled scenario). In addition, we evaluate the quality of the detections by our method considering the efficiency aspect.

We consider that the invention would run on the use case described as the best mode embodiment (1st embodiment) for the experiments executed. Thus, we employed a configuration with a few parameters to reduce battery consumption and low memory restrictions. The final configuration used is composed of: (i) the preprocessor component 107, including data augmenter 201, normalizer 203, hand-laterality detector 204, transformer 205, and segmenter 206; (ii) the feature extractor 108, including the automatically learned feature extractor 302; and (iii) the probability estimator 109, including the meal session probability calculator 402.

Effect on Meal Intake Detection with Inertial Sensors

Although the present invention is not dependent on inertial sensors (i.e., accelerometer and gyroscope), and it could be applied in scenarios where no inertial sensors are provided, we evaluated this invention by comparing it with state-of-the-art methods on the well know and public Free-living Food Intake Cycle (FreeFIC) dataset (by Kyritsis, Konstantinos, Christos Diou, and Anastasios Delopoulos. “A data-driven end-to-end approach for in-the-wild monitoring of eating behavior using smartwatches.” IEEE Journal of Biomedical and Health Informatics, v. 25, n. 1, p. 22-34, 2020). FreeFIC was created by recording the subjects' meals as a small part of their everyday life, unscripted activities. The FreeFIC dataset contains the 3D acceleration and orientation velocity signals (6 DoF) obtained with inertial sensors. All sessions were recorded using a commercial smartwatch while the participants performed their everyday activities. In addition, FreeFIC also contains the start and end moments of each meal session, as reported by the participants.

To assess the prediction performance, we consider the default F1-score metric for comparison on this dataset:

$${F}_{1} = \frac{TP}{TP + \frac{1}{2} ( FP + FN )}$$

where a true positive (TP) happens when the model correctly predicts an eating event, a true negative (TN) is an outcome of the model correctly predicting a non-eating event, and a false positive (FP) happens when the model incorrectly predicts an eating event and a false negative (FN) is an outcome of the model incorrectly predicting a non-eating event.

We also used an in-the-wild dataset from Samsung Research Russia (SRR) composed of 91 subjects captured during all-day use. For such a dataset, we consider the Precision and Recall metrics:

$$Precision = \frac{TP}{TP + FP}$$

$$Recall = \frac{TP}{TP + FN}$$

In Table 1, we present our results compared to previous methods from the literature, as well as our approach considering a convolutional neural network (CNN) and a recurrent neural network (RNN) architecture used as component 108 (see FIG. 1). The proposed approach with CNN improves compared to CNN+LTSM by 1.6 percentage points (p.p.) in the F1-score metric while reducing the model size from 163.6 k to 7.7 k parameters. This demonstrates that the proposed approach allows a compact representation with improved performance. It also confirms the superiority of the present invention when compared to other available approaches.

TABLE 1
Evaluation of our approach for meal intake detection,
on the FreeFIC dataset, compared to previous work.

| Approach | F1-score | # Parameters | |
|---|---|---|---|
| This invention (CNN) | 0.915 | 7.7 | k |
| This invention (RNN) | 0.859 | 25.5 | k |
| CNN + LSTM [1] | 0.899 | 163.6 | k |

| | | |
|---|---|---|
| DBSCAN [1] | 0.865 | — |
| Hysteresis threshold + Naive Bayes [2] | 0.400 | — |

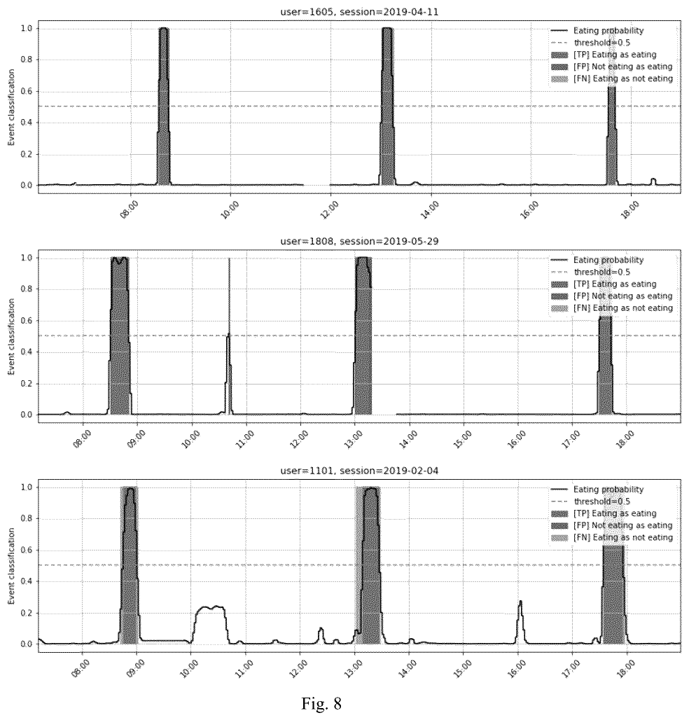

In Table 2, we present the results of this invention using the SRR dataset with our approach considering CNN and RNN architectures used as component 108 (see FIG. 1). Considering the use of this invention with RNN, it achieves 0.920 and 0.838 in precision and recall metrics, respectively. Analyzing directly the recall that measures the errors related to false negatives—i.e., an outcome of the model incorrectly predicting a non-eating event-we note that these errors occur only in the early and final minutes of a meal session (see FIG. 8). Our best model, in this case, presents a model with 20.4 k parameters. This demonstrates that the proposed approach can precisely detect meal intake sessions with low fail cases. The proposed invention also contributes to implementing and using lightweight models (i.e., with fewer parameters) since using the pre-processing component can directly impact the model size.

TABLE 2
Evaluation of our approach for meal
intake detection on the SRR dataset.

| Approach | Precision | Recall | # Parameters |
|---|---|---|---|
| This invention (CNN) | 0.917 | 0.815 | 21.4 k |
| This invention (RNN) | 0.920 | 0.838 | 20.4 k |

In FIG. 8, we present the qualitative results of the present invention. These examples graphically show the very low false positive and false negative cases demonstrating that our approach can deal with real-world meal intake sessions.

The precision metric, as previously defined, refers to the proportion of identifications, i.e., the system suggests that the user is in a meal session, which is correct and expected. So, a model without false positives would have the maximum precision. But even a system with perfect precision could miss some meal sessions. So, it is essential also to analyze the system recall.

The Recall metric refers to the proportion of actual positive classifications that were identified correctly. So, a model without false negatives would lead to a maximum recall.

However, improving the precision metric typically reduces the recall metric and vice versa. So, it is essential to analyze the application context. In the experiments, the precision was consistently higher than the recall, allowing a good user experience that false positives would preclude. Regardless, in the particular context of this invention, the proposed solution can maintain acceptable performance for both metrics.

### Effect of Post-Processing Technique on the Performance of the Whole Method

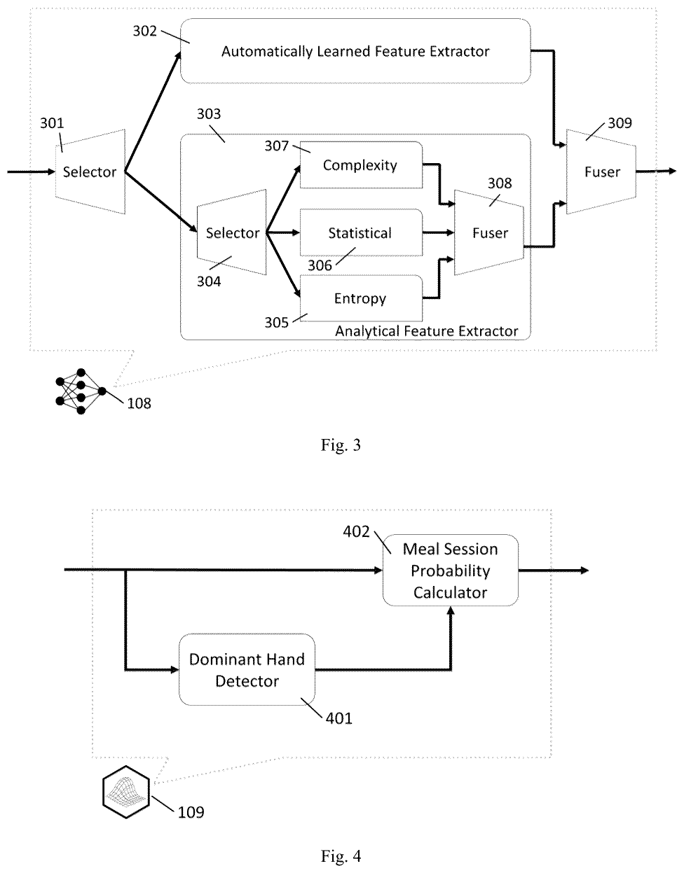

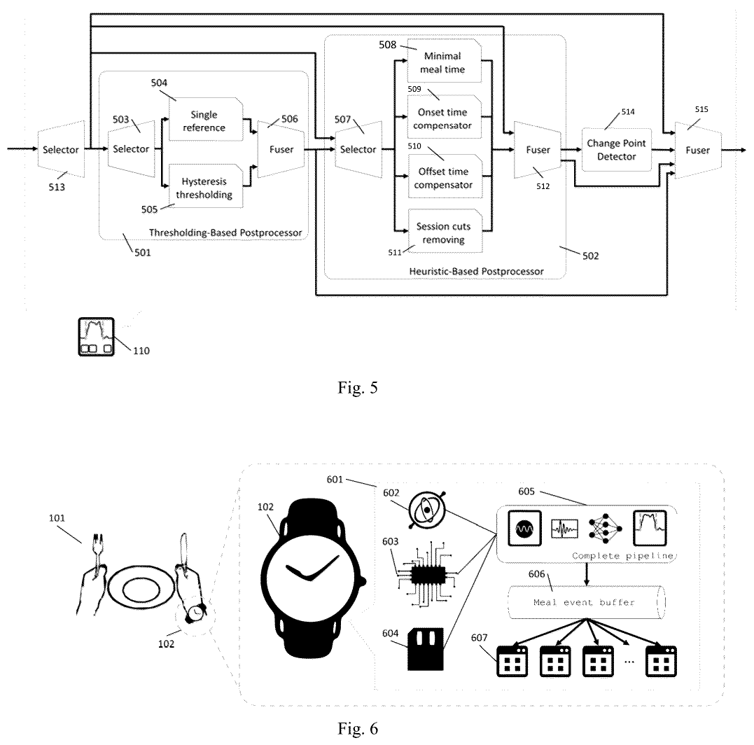

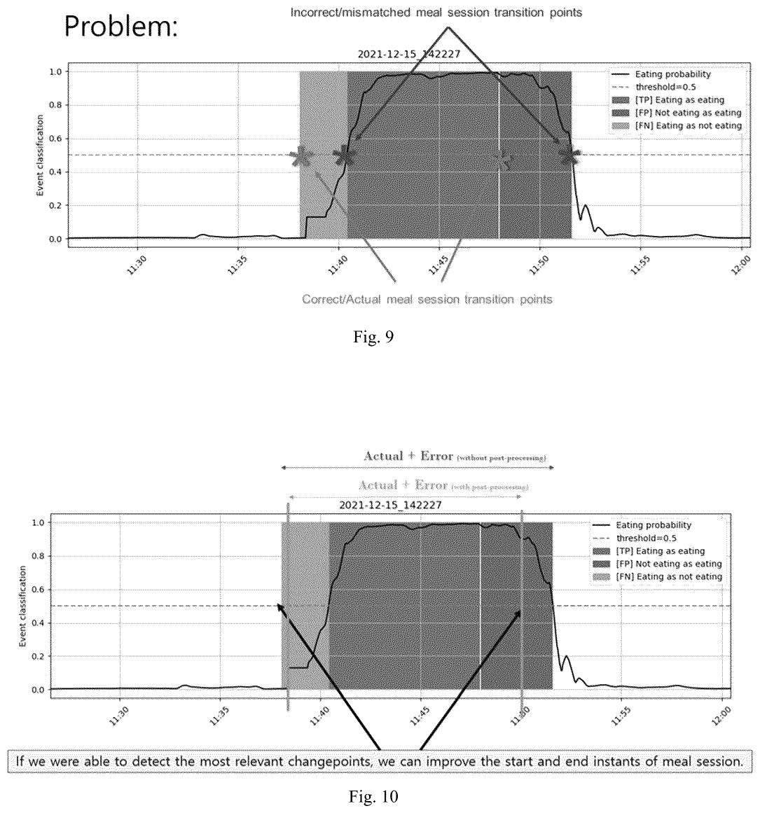

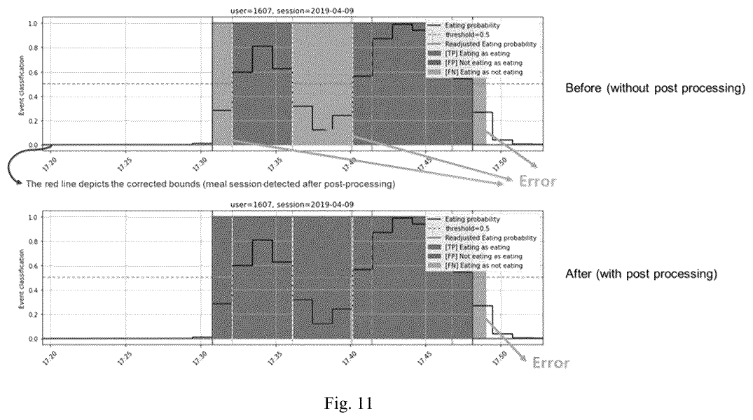

FIG. 9 illustrates the problem that the post-processing component tries to solve. In this figure, the black curve symbolizes the probability curve predicted by the Probability Estimator (see FIGS. 1, 3, and 4). This curve is formed with the probability of a given subject being in a meal intake session as a function of time. The step formed by the probability curve in FIG. 9 illustrates a meal intake session, i.e., when the user starts and finishes eating. In this task, this invention tries to solve the challenge of determining when this change of state (non-eating to eating) started and when it returned to the previous state (eating to non-eating). Thus, transition zones usually have an associated error. In FIG. 9, the pink zones indicate false negative errors (when the Probability Estimator should detect it as eating, and it did not), and the blue zone indicates an accurate prediction (when the user was eating, and the Probability Estimator detected it correctly). The red shows a false positive zone (when the user was no longer eating and the Probability Estimator detected it as if it was). These errors occur because the Probability Estimator predicts the points highlighted with red asterisks as the change points while it should detect the green asterisks. Therefore, the idea behind post-processing is to reduce these transition regions so that the overall error associated with the feed session is minimized, as illustrated in FIG. 10.

FIG. 11 shows another effect of using the post-processing component. The first graph in this figure corresponds to a single meal intake session wrongly predicted as two meal sessions in sequence (notice the multiple blue zones in a short time). This first graph shows a prediction of a meal intake session with no post-processing stages. On the other hand, after applying the post-processing technique, it is noticeable that the erroneous predictions (pink zones) are drastically decreased, indicating that the final prediction was improved.

### Effect on Smartwatch Embedded System for Meal Intake Detection Using Inertial Sensors

To demonstrate the applicability and better understand the present invention's feasibility, a Proof-of-Concept (PoC) application replicating the use case described as the best mode embodiment (1st embodiment) was developed.

The proposed pipeline 605 was ported to the application layer of the smartwatch operative system. This porting is not restricted to deployment to the operative system application layer. It can be fully or partially migrated to a microcontroller, co-processor, digital signal processor (DSP), or any other processing unit (typically a sensor hub) available in the target platform.

The PoC was developed considering sensor 602, the inertial mass unit embedded in the tested platform. This inertial unit consists of three-dimensional acceleration channels and three-dimensional angular velocity channels.

The proposed method 605 uses as pre-processing the automatically learned features block 302, specifically, a convolutional neural network. The dominant hand detector 401 was implemented to be received as additional user input. The probability estimator 402 was implemented as a recurrent neural network. Blocks 302 and 402 correspond to the encoder and decoder on the architecture described in [3]. The preprocessor and the probability estimator can be obtained in a single step. The output probability calculated in 402 is processed by the single reference threshold module 504. The parameters of modules 302, 402, and 504 were optimized simultaneously using the BOHB method [4].

Every meal session detected by this proposed configuration is employed as input to a Meal event buffer 606, where these events are redirected to the notification system and the device storage 607.

The inference time, defined as the time it takes for the whole pipeline to be executed, results in a time of approximately 500 ms when evaluated in a five-minute input window. This is an input-output inference time ratio of 600:1, which could be considered acceptable in an online or real-time application.

The inference interval time, defined as the time between two probability estimations, was set to 60 seconds by default. The inference interval time definition implies a clear trade-off between the user experience and the battery consumption: the lower the inference interval time, the better the user experience but, the higher the battery consumption. As this application was intended to be a PoC, no further resource optimization was conducted.

The results obtained with the implemented application showed no performance loss in terms of the evaluation metrics when compared with the offline model evaluations.

The exemplificative embodiments described herein may be implemented using hardware, software, or any combination thereof and may be implemented in one or more computer systems or other processing systems. Additionally, one or more of the steps described in the example embodiments herein may be implemented, at least in part, by machines. Examples of machines that may be useful for performing the operations of the example embodiments herein include wearable electronic devices, such as smartwatches.

For instance, one illustrative example system for performing the operations of the embodiments herein may include one or more components, such as one or more microprocessors, for performing the arithmetic and/or logical operations required for program execution, and storage media, such as one or more disk drives or memory cards (e.g., flash memory) for program and data storage, and random access memory, for temporary data and program instruction storage.

Therefore, the present invention is also related to a system for detecting food intake comprising a processor and a memory comprising the computer-readable instructions that, when performed by the processor, cause the processor to perform the method steps previously described in this disclosure.

The system may also include software resident on a storage media (e.g., a disk drive or memory card), which, when executed, directs the microprocessor(s) in performing transmission and reception functions. The software may run on an operating system stored on the storage media, such as, for example, UNIX or Windows, Linux, Android, and the like, and can adhere to various protocols such as the Ethernet, ATM, TCP/IP protocols and/or other connection or connectionless protocols.

As well known in the art, microprocessors can run different operating systems and contain different software types, each type being devoted to a different function, such as handling and managing data/information from a particular source or transforming data/information from one format into another format. The embodiments described herein are not to be construed as being limited for use with any particular type of server computer, and any other suitable device for facilitating the exchange and storage of information may be employed instead.

Software embodiments of the illustrative example embodiments presented herein may be provided as a computer program product or software that may include an article of manufacture on a machine-accessible or non-transitory computer-readable medium (also referred to as “machine-readable medium”) having instructions. The instructions on the machine-accessible or machine-readable medium may be used to program a computer system or other electronic device. The machine-readable medium may include, but is not limited to, floppy diskettes, optical disks, CD-ROMs, magneto-optical disks, or another type of media/machine-readable medium suitable for storing or transmitting electronic instructions.

Therefore, the present invention also relates to a non-transitory computer-readable storage medium for detecting food intake from wearable devices, comprising computer-readable instructions that, when performed by the processor, cause the processor to perform the method steps previously described in this disclosure.

The techniques described herein are not limited to any particular software configuration. They may be applicable in any computing or processing environment. The terms “machine-accessible medium,” “machine-readable medium” and “computer-readable medium” used herein shall include any non-transitory medium that is capable of storing, encoding, or transmitting a sequence of instructions for execution by the machine (e.g., a CPU or other type of processing device) and that cause the machine to perform any one of the methods described herein. Furthermore, it is common in the art to speak of software in one form or another (e.g., program, procedure, process, application, module, unit, logic, and so on) as taking action or causing a result. Such expressions are merely a shorthand way of stating that the execution of the software by a processing system causes the processor to act to produce a result.

Therefore, the proposed invention provides a novel method and framework to detect autonomously meal intake events using physiological digital signals embedded in different devices and/or health/fitness applications and have an extensible, configurable, and modular nature, covering robustly multiple use cases and devices. The present invention provides a solution using sophisticated workflow mechanisms, allowing customizable target solutions and configurable nature that allows the implementation of the automatic and manual configuration modes. The present invention is advantageous because the flexible and configurable nature enables the adequate setting of the global optimization target, i.e., the aimed requirements, allowing the production of lightweight, accurate systems. Depending on the applications and available hardware, the stages can be customized to generate various embodiments to achieve the appropriate complexity-precision trade-off.

Moreover, this invention proposes novel signal processing blocks for laterality and hand dominance detection. These blocks can be used to increase the specificity of the application to wrist-worn and wrist-dependent devices and refine the data input required for the detection.

While various exemplary embodiments have been described above, it should be understood that they have been presented by example, not limitation. It is apparent to persons skilled in the relevant art(s) that various changes in form and detail can be made therein.

What is claimed is:

1. A system of detecting food intake events from wearable devices, the system comprising:
a signal bank to store physiological digital data signals collected via a wearable device including at least one sensor to sense at least one physiological parameter of a user;
a preprocessor module to process the physiological digital data signals stored on the signal bank and to create a descriptive representation of the sensed at least one physiological parameter;
a feature extractor module including an automatically learned feature extractor and an analytical feature extractor to obtain features from the descriptive representation, the feature extractor module being configured to automatically select at least one of the automatically learned feature extractor or the analytical feature extractor; and
a probability estimator module to determine whether a food intake event of the user occurs based on the obtain features.

2. The system according to claim 1, wherein the at least one sensor is an inertial sensor, a temperature sensor, a bioelectrical impedance sensor, or a photoplethysmography sensor.

3. The system according to claim 1, wherein the preprocessor module comprises a data augmenter to apply data augmentation on the signal bank in order to create representative data to describe the food intake event.

4. The system according to claim 1, wherein the preprocessor module further comprises:
a filter and a normalizer to adjust a signal received from at least one sensor into a numerical representation, wherein the filter is configured to perform noise reduction, signal smoothing and/or to suppress unwanted signal frequencies, and the normalizer is configured to adjust a range of the signal.

5. The system according to claim 1, wherein the preprocessor module further comprises:
a hand-laterally detector configured to, in case the wearable device is positioned on an arm of the user, detect in which user's arm the device wearable is being used, and
a transformer is configured to transform inertial physiological data based on an output of the hand-laterally detector.

6. The system according to claim 1, wherein the preprocessor module further comprises a segmenter configured to subdivide sampled signals into segments, and the segmenter is further configured to subdivide the segments into windows.

7. The system according to claim 1, wherein the Automatically Learned Feature Extractor comprises a machine learning model configured to extract representative features from the physiological digital data signals.

8. The system according to claim 1, wherein the Analytical Feature Extractor is configured to extract multiple analytical features and is further configured to automatically select at least one of entropy, statistical or complexity features.

9. The system according to claim 1, wherein the probability estimator module further comprises a dominant hand detector configured to detect a dominant hand of the user.

10. The system according to claim 1, further comprising a postprocessor module configured to form a time series on the estimated probability and to output an adjusted probability curve.

11. The system according to claim 10, wherein the postprocessor module is further configured to select at least one of a Thresholding-Based Postprocessor and a Heuristic-Based Postprocessor, wherein the Thresholding-Based Postprocessor is configured to select at least one of reference or hysteresis thresholding, and
the heuristic-based postprocessor is configured to select at least one of a minimal meal time, an onset time compensator, an offset time compensator, and a session cut-removing module.

12. The system according to claim 10, wherein the postprocessor module further comprises a change point detector configured to detect changes in a transition of events.

13. The system according to claim 1, further comprising a logbook for recording the food intake events.

14. The system according to claim 1, further comprising a meal event buffer for storing the detected food intake events.

15. The system according to claim 1, further comprising a data buffer to store data representation created by the preprocessor, wherein the data buffer is connected to another data buffer over a network, and another data buffer stores transmitted data representation.

16. A method of detecting food intake events from wearable devices, the method comprising:
storing physiological digital data signals in a signal bank, the physiological digital data signals being collected via a wearable device including at least one sensor to sense at least one physiological parameter of a user;
processing, with a preprocessor module, the physiological digital data signals stored on the signal bank, and creating a descriptive representation of the sensed at least one physiological parameter;
obtaining, with a feature extractor module, features from the descriptive representation, the feature extractor module including an automatically learned feature extractor and an analytical feature extractor and being configured to automatically select at least one of the automatically learned feature extractor or the analytical feature extractor; and
generating, with a probability estimator module, an estimated probability to determine whether a food intake event of the user occurs based on the obtained features.

17. The method according to claim 16, wherein the preprocessor module further comprises a filter and a normalizer, and the method further comprises:
adjusting, with the filter and the normalizer, a data signal received from at least one sensor into a numerical representation;
performing noise reduction, data smoothing, and suppressing unwanted signal frequencies with the filter; and
adjusting, with the normalizer, a range of the data signal.

18. The method according to claim 16, wherein the preprocessor module further comprises a hand-laterally detector and a transformer, and the method further comprises:
in case the wearable device is positioned on an arm of the user, detecting in which user's arm the device wearable is being used; and
transforming inertial physiological data based on an output of the hand-laterally detector.

19. The method according to claim 16, wherein the Automatically Learned Feature Extractor comprises a machine learning model configured to extract representative features from the physiological digital data signals.

20. The method according to claim 16, wherein the Analytical Feature Extractor is configured to extract multiple analytical features and is further configured to automatically select at least one of entropy, statistical or complexity features.

21. The method according to claim 16, wherein the probability estimator module further comprises a dominant hand detector configured to detect a dominant hand in a feed action of the user.

22. The method according to claim 16, c further comprising:
forming, with a postprocessor module, a time series on the estimated probability and to output an adjusted probability curve.

23. The method according to claim 22, wherein the postprocessor module is further configured to select at least one of a Thresholding-Based Postprocessor and a Heuristic-Based Postprocessor, wherein the Thresholding-Based Postprocessor is configured to select at least one reference or hysteresis thresholding, and
the Heuristic-Based Postprocessor is configured to select at least one of a minimal meal time, an onset time compensator, an offset time compensator, and a session cut-removing module.

24. The method according to claim 16, further comprising detecting, with a postprocessor module which includes a change point detector, changes in a transition of events.

25. A non-transitory computer-readable storage medium storing computer-readable instructions, when performed by a processor, cause a computer to perform the method defined in claim 19.
